(19) United States
(12) Patent Application Publication
(10) Pub. No.: US 2015/0216477 A1
(43) Pub. Date: Aug. 6, 2015

(54) METHODS FOR DETECTING LYMPHEDEMA

(71) Applicants: SAMIR I. SAYEGH, CHAMPAIGN, IL (US); ALPHONSE TAGHIAN, WESTWOOD, MA (US)

(72) Inventors: SAMIR I. SAYEGH, CHAMPAIGN, IL (US); ALPHONSE TAGHIAN, WESTWOOD, MA (US)

(21) Appl. No.: 14/616,443

(22) Filed: Feb. 6, 2015

Related U.S. Application Data
(60) Provisional application No. 61/936,861, filed on Feb. 6, 2014.

(51) Int. Cl. A61B 5/00; A61B 8/08; A61B 5/107; A61B 5/053

(52) U.S. Cl. CPC A61B 5/4878; A61B 5/0077; A61B 5/0537; A61B 8/0858; A61B 5/1079; A61B 5/1073

(57) ABSTRACT

The invention provides methods for detecting lymphedema and methods for managing lymphedema in an individual. The methods of the invention involves utilizing a light sensing device having a video camera, depth sensor and software effective to perform a 3-dimensional scan and generate a three-dimensional digital model of at least a portion of the body from data obtained by the camera and depth sensor, thereby obtaining a three-dimensional digital model of at least a portion of the body from which the size of a select region of the body can be assessed to detect, monitor and/or manage lymphedema. The invention also provides methods to detect and manage lymphedema that involve assessing any combination of size, amount of extracellular fluids, and thickness of the skin at a select region of the body of an individual.

### CROSS-REFERENCE TO RELATED APPLICATIONS

This application claims priority to U.S. Provisional Patent Ser. No. 61/936,861 filed Feb. 6, 2014, which is incorporated herein by reference in its entirety.

### BACKGROUND

Lymphedema is swelling in regions of the body that include the upper and lower extremities and can occur in individuals who have undergone cancer treatment. Lymphedema is caused by abnormal buildup of protein-rich fluids in the interstitial space resulting from inadequate lymphatic drainage. Upper extremity lymphedema, for example, has been documented in patients who have undergone treatment for breast cancer. Lymphedema can have a profound impact on patient health as it can lead to restricted range of motion, aching or discomfort, recurring infections and fibrosis of the skin. Although there is no cure for lymphedema, early diagnosis can have a significant impact on management of the progression of lymphedema and the associated discomfort and loss of function.

### SUMMARY OF THE INVENTION

The invention provides methods for detecting and/or managing lymphedema. The methods of the invention utilize a light-sensing device that has a video camera, depth sensor and software for generating a three-dimensional, digital model of at least a portion of the body of an individual from three-dimensional scanning data obtained by the camera and depth sensor to obtain a three-dimensional digital model of at least a portion of the body of the individual, from which the size of one or more select regions of the body can be determined. The methods for detecting lymphedema can utilize size, or any combination of size, amount of extracellular fluids, and skin thickness to detect lymphedema in a select region of the body. The invention also provides methods for managing lymphedema that involves monitoring for lymphedema and providing medical information, guidance or treatment to manage lymphedema.

In one aspect, the invention provides a method for detecting lymphedema in an individual that involves: (a) using a light sensing device with a video camera, depth sensor and software for generating a three-dimensional digital model of at least a portion of the body of the individual from data obtained by the camera and depth sensor to obtain a three-dimensional digital model of at least a portion of the body of the individual; (b) determining the size of a first select region of the body from the three-dimensional digital model; and (c) utilizing the size of the first select region and the size of a first reference region similarly determined according to steps (a) and (b) to obtain a size differential, wherein lymphedema is detected in the first select region if the size differential indicates that the size of the first select region is greater than the size of the first reference region by at least about 3%.

In another aspect, the invention provides a method for detecting lymphedema in an individual that involves determining a size differential for a first select region and a first reference region according to a method of the invention described, determining the amount of extracellular fluids in the first select region of the body of the individual using bioimpedance spectroscopy, and comparing the amount of extracellular fluids in the first select region with the amount of extracellular fluids in the first reference region similarly determined using bioimpedance spectroscopy to obtain an impedance ratio change, wherein lymphedema is detected in the first select region if the size differential indicates that the size of the first select region is greater than the size of the first reference region by at least about 3%, and the impedance ratio change is about 0.1 or more.

In another aspect, the invention provides a method for detecting lymphedema in an individual that involves determining a size differential for a first select region and a first reference region according to a method of the invention described herein, determining skin thickness in the first select region using ultrasound, and comparing the thickness of the skin in the first select region with thickness of the skin in the first reference region similarly determined using ultrasound to obtain an impedance ratio change, wherein lymphedema is detected in the first select region if the size differential indicates that the size of the first select region is greater than the size of the first reference region by at least about 3%, and thickness of the skin is increased by at least about 5%.

In another aspect, the invention provides a method for detecting lymphedema in a portion of the body of an individual that involves determining the amount of extracellular fluids and skin thickness at a select region of the body of the individual, wherein amount of extracellular fluids is determined using bioimpedance spectroscopy, and thickness of the skin is determined using ultrasound; and comparing the amount of extracellular fluids and skin thickness at the select region to that of a reference region similarly determined according to step (a), wherein early-stage lymphedema is detected in the select region if the increase in amount of extracellular fluids in the select region relative to the reference region corresponds to an impedance ratio change of about 0.1 or more, and the skin thickness at the select region relative to the reference region is increased by at least about 5%.

In some embodiments of a method of the invention, the light sensing device includes a RGB video camera and a charge-coupled device (CCD) image sensor, contact image sensor (CIS), or an active pixel sensor in complementary metal-oxide-semiconductor (CMOS). In some embodiments, the light sensing device is a mobile device having software for generating, in real time, a three-dimensional model of at least a portion of the body of an individual.

In some embodiments of a method of the invention, the sizes of the first select region and first reference region are indicated by the width, circumference or volume of the regions. In some embodiments, the first select region and first reference region include symmetrical portions of the left and the right extremities.

In some embodiments, the size differential corresponds to the difference between the size of the first select region and the size of the first reference region. In some embodiments, the first select region and first reference region include substantially the same portion of the body occurring at a post-surgery and a pre-surgery time point, respectively. In some embodiments, the first select region and first reference region include substantially the same region of the body occurring two time points post surgery, a later time point and an earlier time point, respectively.

In some embodiments of a method of the invention, size differential is weight adjusted. In some embodiments, the method includes determining the individual's weight at the post-surgery time point, and further utilizing the individual's weight at the post-surgery time point and a reference weight of the individual similarly determined at the pre-surgery time point to obtain a weight adjusted size differential, wherein lymphedema is detected in the first select region if the size differential indicates that the size of the first select region is greater than the size of the first reference region by at least about 3%. In some embodiments, the weight adjusted size differential is determined from the weight adjusted volume change equation:

WAC=[(A₂W₁)/(W₂A₁)]−1

wherein A₁ and A₂ are sizes of the select region determined at pre- and post-surgery time point, respectively, and W₁ and W₂ correspond to the individual's weight determined at the pre- and post-surgery time point, respectively.

In other embodiments of a method of the invention, the method includes determining the size of a second select region of the body from the three-dimensional digital model, wherein the first and second select regions include symmetrical portions of the left and the right extremities, and further utilizing the size of the second select region and a similarly determined size of a second reference region to obtain a size differential that corresponds to a relative volume change, wherein the second reference region corresponds substantially to the same portion of the body as the second select region and occurring at the pre-surgery time point. In some embodiments, the size differential is determined from the relative volume change equation:

RVC=[(A₂U₁)/(U₂A₁)]−1

wherein A₁ and A₂ are sizes of a first reference region and a first select region determined at a pre- and post-surgery time point, respectively, and U₁ and U₂ are sizes of a second reference region and second select region determined at the same pre- and post-surgery time points, respectively.

In some embodiments of the invention in which skin thickness is determined, skin thickness can be indicated by epidermis-dermis depth, subcutis depth, or both epidermis-dermis depth and subcutis depth.

In another aspect, the invention provides a method for managing lymphedema in an individual that involves monitoring for lymphedema in an individual at risk for lymphedema using any method of the invention described herein and providing medical guidance or treatment to manage lymphedema in the individual.

Any feature or combination of features described herein are included within the scope of the present invention provided that the features included in any such combination are not mutually inconsistent as will be apparent from the context, this specification and the knowledge of one of ordinary skill in the art.

Unless otherwise defined, all technical and scientific terms used herein have the same meaning as commonly understood by one of ordinary skill in the art to which this invention belongs. In case of conflict, the present specification, including definitions, will control. In addition, the materials, methods, and examples are illustrative only and not intended to be limiting. Although methods and materials similar or equivalent to those described herein can be used to practice the invention, suitable methods and materials are described below.

All patents and publications referenced or mentioned herein are indicative of the levels of skill of those skilled in the art to which the invention pertains, and each such referenced patent or publication is hereby incorporated by reference to the same extent as if it had been incorporated by reference in its entirety individually or set forth herein in its entirety. Applicants reserve the right to physically incorporate into this specification any and all materials and information from any such cited patents or publications.

Other features and advantages of the invention will be apparent from the following detailed description and from the claims.

### BRIEF DESCRIPTION OF THE FIGURES

FIG. 1 is a three-dimensional, digital model of the trunk of an individual obtained using a light-sensing device such as KINECT, the model having linear differences within 2 mm and volume consistent with repeatability of manual technique.

### DETAILED DESCRIPTION OF THE INVENTION

The invention provides methods for detecting lymphedema and methods for managing lymphedema in an individual. The methods for detecting lymphedema utilize a light-sensing device that has a video camera, depth sensor and software for generating a three-dimensional, digital model of at least a portion of the body of an individual from three-dimensional scanning data obtained by the camera and depth sensor to obtain a three-dimensional digital model of at least a portion of the body of the individual, from which the size of one or more select regions of the body can be determined. The methods for detecting lymphedema can utilize size, or any combination of size, amount of extracellular fluids, and skin thickness to detect lymphedema in a select region of the body. The invention also provides methods for managing lymphedema that involves monitoring for lymphedema and providing medical information, guidance or treatment to manage lymphedema.

As used herein, the phrase “detecting lymphedema” means detecting or diagnosing the existence of lymphedema, as well as the onset of lymphedema, early stage lymphedema and the progression of lymphedema over time. Thus, methods for detecting lymphedema can be used to monitor the progression of lymphedema over time and manage lymphedema in an individual. As used herein, the term “manage” or “managing” lymphedema means providing medical information, guidance and/or treatment to an individual in order to control swelling or control one or more lymphedema-associated discomforts or loss of function. Thus, as used herein, the term “managing lymphedema” or “manage lymphedema” means providing information or guidance related to one or more lymphedema treatment options, as well as providing one or more lymphedema treatment options including, for example, pressure garments, exercise, bandages, skin care, combined therapy, compression device, weight loss, laser therapy, drug therapy, surgery and massage therapy.

The methods of the invention involve assessing one or more modalities including size, amount of extracellular fluids and skin thickness. The methods of the invention involve (1) determining size from a three-dimensional digital model of at least a portion of the body of an individual; or (2) determining any combination of (a) size from a three-dimensional digital model of at least a portion of the body of an individual, (b) amount of extracellular fluids from bioimpedance spectroscopy, and (c) skin thickness determined using ultrasound.

Where a method of the invention employs two or more modalities for detecting lymphedema, for example, size and amount of extracellular fluids, size and skin thickness, amount of extracellular fluids and skin thickness, or size, amount of extracellular fluids and skin thickness, the modalities are assessed concurrently. As used herein, the term “concurrently” means substantially at the same time, in sequence in as close temporal relation as is practical so as to minimize elapsed time therebetween, or within the same clinic visit or within the same day so as to minimize the effects of temporal changes on the second modality to be assessed. Thus, two or more modalities are assessed concurrently when they are assessed substantially at the same time, in sequence in as close temporal relation as is practical, or within the same clinic visit or same day. For example, where size and amount of extracellular fluids are assessed, the three-dimensional digital model is obtained and bioimpedance spectroscopy performed in sequence and in as close temporal relation as is practical or possible to minimize temporal changes in the observed impedance ratio. Similarly, where size and skin thickness are assessed, the three-dimensional, digital model is obtained and ultrasound imaging of the skin performed concurrently or in sequence in as close temporal proximity as is practical or possible. Where amount of extracellular fluids and skin thickness are assessed, bioimpedance spectroscopy and ultrasound are performed concurrently or in close sequence thereby allowing fluid amounts and skin thickness occurring at the substantially the same time to be assessed.

The methods of the invention involve determining the magnitude of a change or difference in the one or more modalities exhibited by a select region of the body of an individual relative to a reference region. The term “select region” and “reference region” can refer to different regions of the body, for example, symmetrical or matched regions on the upper or lower extremity, as well as the same region of the body, for example, the neck or trunk, occurring at two different time points, for example, pre-surgery and post-surgery, or occurring at two different time points post-surgery.

Where the terms “select region” and “reference region” denote different regions of the body, the select region can be a region or portion of the body that is ipsilateral to the site of surgery, as this region or portion of the body is at-risk for developing lymphedema. For example, where an individual has had unilateral surgery, the region or portion of the body ipsilateral to the site of surgery is selected for lymphedema monitoring as it is at-risk for developing lymphedema. In these embodiments, the reference region can be the corresponding region or portion of the body that is contralateral to the site of surgery. As such, the select, at-risk region and reference region can be symmetrical or matched regions on the left and right portion of the upper or lower extremity.

In some embodiments, the select region and reference region can be substantially the same region of the body occurring pre-surgery and post-surgery. For example, where an individual has had bilateral surgery, the region of the body selected for analysis can be substantially the same as the reference region, and the term “select region” and “reference region” refer to the same region of the body, e.g. trunk of the body, occurring post-surgery and pre-surgery, respectively.

In other embodiments, the select region and reference region can be substantially the same region of the body occurring at different time points post-surgery. For example, the select region can be on the trunk of the body at one time point post surgery, and the reference region can be at substantially the same position of the trunk at an earlier time point. Similarly, the select region can be on the right upper extremity at a select time point post-surgery, and the reference region can be substantially the same region on the right upper extremity at an earlier time point post-surgery. As such the methods of the invention allow for monitoring or detecting the onset of lymphedema, early stage lymphedema or progression of lymphedema by assessing the change in one or more modalities over a period of time. The time points at which the various lymphedema modalities can be assess can be any two time points including time points that are pre- and post-surgery, as well as any two time points post-surgery. The time points can be, without limitation, about 1 hour, about 2 hours, about 3 hours, or more than about 3 hours apart, as well as about 1 day apart, about 2 days apart, about 3 days apart, about week a part, about two weeks apart, about three weeks apart, about month a part, about two months apart, about four months apart, about five months apart, about six months apart or more than about six months apart. Thus, the methods of the invention allow for monitoring or detecting lymphedema, the onset of lymphedema or early stage lymphoma at any two or more time points including time points that are pre- and post-surgery, or time points that are post surgery.

Thus, a change in one or more modalities can be determined by assessing the modalities at a select region before and after surgery, and a difference or differential in one or more modalities between a select region, e.g. at-risk region, and a reference region can be determined by assessing the modalities at symmetrical or matched regions on the left and right side of the body or the left and right extremities.

Size and Size Differential

The methods of the invention involve determining size or any combination of size, amount of extracellular fluids and skin thickness.

The size of a region of the body of an individual can be indicated by its width, circumference or volume. For example, the size of the foot can be based on the width of the foot, its circumference or volume. The size of a region of the body can be determined by direct measurement using a measuring device including, for example, tape measure, dial caliper and volumeter. More specifically, a tape measure can be used to measure the girth of each arm or hand. Similarly, the arm can be measure from the wrist up the arm to the axilla at specific landmarks or segments. Dimensions such as length, width, depth, circumference, girth and volume can be measured directly or through water displacement. More specifically, the arm can be placed into a cylinder of water and arm volume is determined based on the amount of water displaced by the arm. Alternatively, volume and surface area can be computed from one or more of these dimensions. See, for example, Sander et al., Upper-extremity Volume Measurements in Women with Lymphedema: A Comparison of Measurements Obtained via Water Displacement with Geometrically Determined Volume, Physical Therapy 82:1201-12 (2002), the contents of which are in incorporated herein by reference in their entirety.

The size of a region of the body of an individual can also be determined from a three-dimensional, digital model of at least a portion of the body of an individual that is obtained using a light sensing device having a video camera, depth sensor and software capable of generating a three-dimensional, digital model of a three-dimensional object from 3-dimensional scanning data obtained by the video camera and depth sensor. Thus, a light sensing device that can be used in a method of the invention include a video camera and a depth sensor for scanning and capturing 3-dimensional data related to the structure and dimension of a three-dimensional object such as a portion of the body of an individual. The camera can be a red-green-blue (RGB) camera that obtains and stores three channel data, for example, at 30 frames per second, in 1280×960 resolution or 640×480 pixel resolution. The depth sensor can be a combination of infrared (IR) emitor or projector and an IR depth sensor, for example, a monochrome complimentary metal-oxide semiconductor (CMOS) for obtaining depth information. A device of the invention can also be a time-of-flight camera with time-of-flight sensing function that allows for direct measurement of depth and amplitude in each pixel. See, for example, Kadambi et al. Coded Time of Flight Cameras: Sparse Deconvolution to Address Multipath Interference and Recover Time Profiles, ACM Transactions on Graphics—Proceedings of ACM SIGGRAPH Asia 32(6), Article 167, November 2013, available at http://web.media.mit.edu/˜achoo/lightsweep/paper.pdf (retrieved Feb. 6, 2015); and Li, Time-of-Flight Camera—An Introduction, Technical White Paper, SLOA190B—January 2014, Revised May 2014, Texas Instruments (available at http://www.ti.com/lit/wp/sloa190b/sloa190b .pdf, retrieved Feb. 6, 2015). Examples of time-of-flight cameras include the devices with RF-modulated light sources with phase detectors, range gated imagers, and direct time-of-flight imagers. The light sensing device for use in a method of the invention also includes software for generating a three-dimensional digital model of a three-dimensional object from 3-dimensional scanning data related to the structure and dimension of the object that is obtained from the camera and depth sensor.

Light sensing devices are known to those of skilled in the art, and non-limiting examples include non-contact profilometers, white light interferometer, laser profilometers, red-green-blue (RGB) cameras with depth sensors, Microsoft's KINECT (see, for example, Kinect for Xbox One, http://www.xbox.com/en-US/xbox-one/accessories/kinect-for-xbox-one, last visited Feb. 3, 2015), Asus Xtion and Primesense Carmine (Primesense, Apple Inc., Cupertino, Calif., US), a three-dimensional scanner such as Structure Sensor with software such as Skanect (Occipital, Inc., San Francisco, Calif.), as well as a mobile device such as a computer tablet in combination with Structure Sensor or Room Capture (Occipital, Inc., San Francisco, Calif.). Additional light sensing devices with depth sensing capability include time-of-flight imagers known to those of skilled in the art including, for example Infineon 3D Image Sensor IRS10x0C (Infineon Technologies AG, Munich, Germany).

Light sensing devices are combined with image processing software for data acquisition and computation including surface analysis, mapping, dimensional analysis including surface area and volume, as well as statistical analysis and imaging. Thus, from the three-dimensional digital model, the width, circumference or volume of a select region of the body can be measured and computed. For example, the width, circumference or volume of a select region of the body can be measured and computed using known software including, without limitation, KScan3D (LMI Technologies Inc., Delta, B.C. Canada); Microsoft Kinect for Windows SDK 2.0 (Microsoft, Redmond, Wash., US); and Skanect (Occipital, Inc., San Francisco, Calif., US).

Additional examples of methodologies that can be used to determine size of at least a portion of the body of an individual include those described in U.S. Pat. No. 8,150,142, U.S. Pat. No. 8,050,461, and U.S. Pat. No. 4,802,759, as well as the structured light techniques described in Agin, G., Computer Description of Curved Objects, IEEE T. Comput. C-25(4), 439-449 (1976); Chen et al., Overview of Three-dimensional Shape Measurement Using Optical Methods, Opt. Eng., 39:10-22 (2000); and Jing et al., Scanning 3D Full Human Bodies Using Kinects, IEEE T. Vis. Comput. Gr., 18:643-650 (2012), the contents of which are herein incorporated by reference in their entirety.

Where size is the modality used to detect or monitor lymphedema, the size of a select region and the size of a reference region can be used to determine a size differential from which a lymphedema can be detected.

In some embodiments, size differential can be the difference between the size of a select region and the size of the reference region. In these embodiments, the reference region can correspond to a symmetrical or matched region on the other extremity or substantially same region occurring earlier in time, for example a pre-surgery time point or an earlier post-surgery time point. In these embodiments, a size differential indicative of lymphedema can be an increase in volume of at least about 3%, for example, about 4%, about 5%, about 6%, about 7%, about 8%, about 9%, about 10% or more than about 10%. In some embodiments, a size differential indicative of lymphedema in the arm can be an increase in volume by about 200 mL or more or an increase in circumference of about 2 cm or more, as known to those of skilled in the art. See, for example, Ancukiewicz et al., Comparison Of Relative Versus Absolute Arm Size Change As Criteria For Quantifying Breast Cancer-Related Lymphedema: The Flaws In Current Studies And Need For Universal Methodology, Breast Cancer Res. Treat. 135:145-52 (2012).

In some embodiments, the size differential can be a weight-adjusted value determined using the weight adjusted arm volume change (WAC) equation:

WAC=[(A₂W₁)/(W₂A₁)]−1

in which A₁ and A₂ are sizes of a particular body region determined at two time points, a pre-surgery and a post-surgery time point, respectively, or a first post-surgery time point and a second, later post-surgery time point, respectively, and W₁ and W₂ correspond to the individual's weight determined at the two time points, respectively. A description of the weight adjusted arm volume change equation can be found in Miller C L, Specht M C, Horick N, Skolny M N, Jammallo L S, O'Toole J, Taghian A G., A Novel, Validated Method To Quantify Breast Cancer-Related Lymphedema (BCRL) Following Bilateral Breast Surgery, Lymphology 46:64-74 (2013). Thus, the WAC equation can be used where the reference region and select region correspond to substantially the same region occurring at two time points, a pre-surgery time point and a post-surgery time point, respectively, or an earlier post-surgery time point and a later post-surgery time point, respectively. In these embodiments, a size differential indicative of lymphedema can be an increase in volume of at least about 3%, for example, about 4%, about 5%, about 6%, about 7%, about 8%, about 9%, about 10% or more than about 10%.

In some embodiments, the size differential can correspond to a relative volume change value determined using the following relative volume change (RVC) equation:

RVC=[(A₂U₁)/(U₂A₁)]−1

in which A₁ and A₂ are sizes of a first select region and a first reference region determined at a pre- and post-surgery time point, respectively, and U₁ and U₂ are sizes of a second select region and second reference region determined at the same pre- and post-surgery time points, respectively. A description of the relative volume change equation can be found in Ancukiewicz M, Russell T A, Otoole J, Specht M, Singer M, Kelada A, Murphy C D, Pogachar J, Gioioso V, Patel M, Skolny M, Smith B L, Taghian A G., Standardized Method For Quantification Of Developing Lymphedema In Patients Treated For Breast Cancer, Int J Radiat Oncol Biol Phys. 79(5):1436-43 (2011). In these embodiments, the first and second select regions can correspond to symmetrical or matched regions on the left and right extremities occurring at a post-surgery time point, while the first and second reference regions corresponding to the same symmetrical or matched regions on the left and right extremities occurring at a pre-surgery time point. Alternatively, the first and second select regions can correspond to symmetrical or matched regions on the left and right extremities occurring at a later post-surgery time point, while the first and second reference regions corresponding to the same symmetrical or matched regions on the left and right extremities occurring at an earlier post-surgery time point. In these embodiments, a size differential indicative of lymphedema can be an increase in volume of at least about 3%, for example, about 4%, about 5%, about 6%, about 7%, about 8%, about 9%, about 10% or more than about 10%.

Extracellular Fluids

The amount of extracellular fluids (ECF) can be determined using methods known to those of skilled in the art including bioimpedance spectroscopy, which utilizes an electrical current to determine the amount of ECF present. ECF volumes can be determined using whole body wrist-to-ankle bioimpedance spectroscopy (wBIS) and ECF volumes at selected regions of an individual's body, for example, trunk or segments of the upper or lower extremity, can be determined using segmental bioimpedance spectroscopy (sBIS). Methods of performing bioimpedance spectroscopy are know to those of skilled in the art, for example, as described in Zhi et al., Segment-specific Resistivity Improves Body Fluid Volume Estimates from Bioimpedance Spectroscopy in Hemodialysis Patients, J. Appl. Physiol 100:717-724 (2006); Warren et al., The Use of Bioimpedance Analysis to Evaluate Lymphedema, Ann. of Plast. Surg. 58:541-43 (2007); and Kyle et al., Bioelectrical Impedance analysis—Part 1: Review of Principles and Methods, Clinical Nutrition 23:1226-43 (2004). Bioimpedance devices that can be used to practice a method of the invention are known to those of skilled in the art and include Imp SFB7 or Imp XCA (ImpediMed, Brisbane, Australia) as further discussed below.

The amount of ECF is indicated by impedance to current flow, with reduced impedance values indicating lymphedema. Impedance values can be determined for a select region and a reference region and the values compared and expressed as an impedance ratio, that is, the ratio of impedance to current flow between the select region and the reference region. For example, the impedance to current flow in a particular region that occurs post-surgery (i.e., select region) can be compared to the impedance to current flow in the same region that occurred pre-surgery (reference region) to obtain an impedance ratio. The impedance to current flow in a particular region on the ipsilateral side of the body (i.e., select region) can be compared to the impedance to current flow in the symmetrical or matched region on the contralateral side of the body (reference region) to obtain an impedance ratio. Thus, a change in the amount of ECF can be detected by comparing the impedance value of a particular region occurring post-surgery with that occurring pre-surgery (a previously established threshold) to obtain an impedance ratio, which can also be obtained by comparing impedance values of symmetrical or matched portions of the extremities. The change reflected in the impedance ratio can indicate lymphedema, early stage lymphedema or the progression of lymphedema. More specifically, an impedance ratio of less than about 1, for example, about 0.9 or less, which corresponds to an impedance change or difference between two regions of about 0.1 or greater, indicates lymphedema, early stage lymphedema or progression of lymphedema. Thus, lymphedema, early stage lymphedema or lymphedema progression is indicated where the change in the impedance ratio is about 0.1, about 0.15, about 0.2, about 0.25, about 0.3 or more.

Where impedance is measured using a BIS device that yields an L-Dex ratio, an L-Dex ratio of greater than about +7.1 in combination with a second modality indicates lymphedema, early stage lymphedema or progression of lymphedema. More specifically, where a method of the invention involves a two-modality approach that includes size differential and bioimpedance, a size differential of about 3% or more in combination with an L-Dex ratio of greater than about +7.1 indicates lymphedema. In addition, a size differential of about 3% or more in combination with a change in the impedance ratio of about 0.1 or more also indicates lymphedema.

Skin Thickness

Skin thickness can be used in combination with another modality for detecting lymphedema, early stage lymphedema or the progression of lymphedema. Skin thickness can be indicated by the thickness of the epidermal-dermal layer, the thickness of subcutis or the thickness of both. Skin thickness can be measured using ultrasound as known to those of skilled in the art. See for example, Tassenoy et al., Postmastectomy Lymphoedema: Different Patterns of Fluid Distribution Visualized by Ultrasound Imaging Compared with Magnetic Resonance Imaging, Physiotherapy 97: 234-243 (2011); Van Der Veen et al., A Key to Understanding Postoperative Lymphoedema: A Study on the Evolution and Consistency of Oedema of the Arm Using Ultrasound Imaging, The Breast 10:225-230 (2001); Mellor et al., Dual-Frequency Ultrasound Examination of Skin and Subcutis Thickness in Breast Cancer-Related Lymphedema, The Breast Journal 10:496-503 (2004); and Naouri et al., High-Resolution Cutaneous Ultrasonography to Differentiate Lypoedema from Lymphoedema, British Journal of Dermatology 163:296-301 (2010), the contents of which are incorporated herein in their entirety.

Skin thickness can be determined for at least a portion of the body including the extremity such as, for example, the foot, thigh, lower leg, ankle, upper arm, forearm, shoulder region. Skin thickness can be determined for a select region and a reference region and the values compared and expressed as a percent change. Percent increase can be determined by comparing skin thickness in a particular region occurring post-surgery (i.e., select region) with skin thickness in the same region that occurred pre-surgery (reference region). Percent increase can also be determined by comparing skin thickness in a particular region on the ipsilateral side of the body (i.e., select region) with that in the symmetrical or matched region on the contralateral side of the body (reference region). The percent increase in skin thickness can indicate lymphedema, early stage lymphedema or the progression of lymphedema. More specifically, a statistically significant increase of any amount can indicate lymphedema, early stage lymphedema or the progression of lymphedema. For example, a statistically significant increase in epidermal-dermal thickness, subcutis thickness or the thickness of both by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100% or more indicates lymphedema, early stage lymphedema or progression of lymphedema.

Where a method of the invention involves a two-modality approach that includes skin thickness for detecting lymphedema, early stage lymphedema or the progression of lymphedema, the threshold for a positive diagnosis can include a statistically significant increase in skin thickness of about 5%. For example, wherein skin thickness is used in combination with size differential, a statistically significant skin thickness of about 5% in combination with a size differential of about 3% indicates lymphedema, early-stage lymphedema or lymphedema progression. In addition, wherein skin thickness is used in combination with ECF amounts, a statistically significant skin thickness of about 5% in combination with a change in the impedance ratio of about 0.1 or more, or in combination with an L-Dex ratio greater than about +7.1 indicates lymphedema, early-stage lymphedema or lymphedema progression.

Embodiments of the invention are described in the following examples, which do not limit the scope of the invention described in the claims.

### EXAMPLES

### Example 1

### Light Sensing Devices

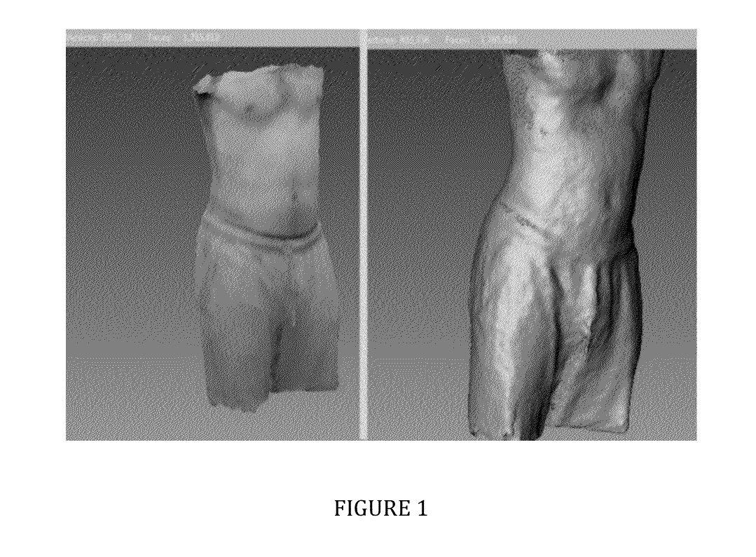

KINECT (Microsoft Corporation, Redmond, Wash.) was used to perform a three-dimensional scan of at least a portion of the body of an individual, for example, an extremity or the neck, trunk or entire body to obtain a three-dimensional digital model of an extremity or the neck, trunk or entire body as shown in FIG. 1. Methods similar to that described in Jing et al., Scanning 3D Full Human Bodies Using Kinects, IEEE T. Vis. Comput. Gr. 18:643-650 (2012) were used. In addition, one or more KINECT devices can be used in a scanning system in which the devices rotate around a stationary individual or the individual is rotated, for example using a turntable, before one or more devices as described in Jing et al. The individual is positioned about 1 meter from one or more devices, the devices or individual rotating about 360° in about 30 seconds. 1280×1024 color images and 640×480 depth images are acquired at 15 frames per second, and three-dimensional coordinates are generated using OpenNI (Occipital, Inc., San Francisco, Calif., U.S.A.).

A mobile device such as a cell phone or tablet with Structure Sensor (Occipital Inc., San Francisco, Calif.) can be used to scan at least a portion of the body of an individual to obtain a three-dimensional digital model in real time from which the size of a particular region or portion as indicated by width, circumference or volume is obtained.

### Example 2

### Bioimpedance Spectroscopy (BIS)

To obtain BIS measurements using Imp SFB7, electrodes are positioned at select positions of an individual's body, for example, at select positions on the upper or lower extremities. Identical electrode positions are used on the left and right extremities to allow comparison of bioimpedance at the left and right extremities. Impedance is calculated using the ImpediMed's software. Statistical analysis is performed using Student's 2-tailed t test where needed. The difference between the left and right extremities can be expressed as a ratio.

BIS can also be performed using the Imp XCA, which employs a single frequency below 30 kHz to measure impedance and resistance of the ECF. Methods known to those of skilled in the art can be used. For example, an individual is placed in a fully supine position with the legs not touching and the arms extended 30° from the body by their sides. Two dual-tab electrodes are placed on the dorsum of the right and left wrists adjacent the ulnar styloid process extending to just proximal to the third metacarpophalangeal joint of the dorsum of the hands; one dual-tab electrode is placed on anterior to the right ankle joints between the malleoli, extending to the dorsum of the right foot over the third metatarsal bone just proximal to the third metatarsophalangeal joint. An impedance ratio relative to normative standards derived from healthy individuals is used to calculate a Lymphedema Index (L-Dex ratio) ratio ranging from −10 to +10. The L-Dex ratio takes into consideration the ratio between dominant and non-dominant arms, which is equivalent to impedance ratios of 0.935 to 1.139 for at-risk dominant arms and 0.862 to 1.066 for at-risk non-dominant arms. As such, an individual is determined to have arm lymphedema or swelling if the individual's L-Dex ratio exceeds +7.1, for example, +10.

A multi-frequency device such as HYDRA ECF/ICF Model 4200 (Xitron Technologies, Inc., San Diego, Calif., US) can be used to obtain automatic sequential BIS measurements of the upper and lower extremities including the trunk of the body at frequencies ranging from 5 KHz to 1 MHz. Current is injected through two electrodes placed on the wrist and the ipsilateral ankle, and voltage is recorded using four electrodes on the wrist and ipsilateral shoulder, greater trochanter and ankle Measurements are repeated multiple times and the average, for example, of ten measurements, is used in the analysis.

### Example 3

### Ultrasound Imaging

Skin thickness can be determined by ultrasound imaging using systems known to those of skilled in the art including, for example, a Sonoline Antares ultrasound system (Siemeans, Erlanger, Germany), an Aloka SSD-1700 Diagnostic Ultrasound System (Aloka Co., Ltd., Wallingford, Conn.), DermaScan C ultrasound scanner (Cortex Technology, Smedevaenget, Denmark), and Dermcup 2020 (Atys Medical, Soucieu en Jarrest, France). Images can be obtained and analysed using methods known to those of skilled in the art, for example, see Tassenoy et al., Postmastectomy Lymphoedema: Different Patterns of Fluid Distribution Visualized by Ultrasound Imaging Compared with Magnetic Resonance Imaging, Physiotherapy 97: 234-243 (2011); Van Der Veen et al., A Key to Understanding Postoperative Lymphoedema: A Study on the Evolution and Consistency of Oedema of the Arm Using Ultrasound Imaging, The Breast 10:225-230 (2001); Mellor et al., Dual-Frequency Ultrasound Examination of Skin and Subcutis Thickness in Breast Cancer-Related Lymphedema, The Breast Journal 10:496-503 (2004); and Naouri et al., High-Resolution Cutaneous Ultrasonography to Differentiate Lypoedema from Lymphoedema, British Journal of Dermatology 163:296-301 (2010), the contents of which are incorporated herein in their entirety.

To assess the skin thickness at select portions of the upper extremity of an individual, ultrasound gel is applied to the skin at the region of interest and images are obtained using a probe placed perpendicular to the skin. A Sonoline Antares ultrasound system available through Siemens (Erlanger, Germany) can be used with a 10 MHz transducer. For assessment of the arm, for example, images from the front, back, and/or sides of the arm is obtained by placing the probe transversely relative to the longitudinal axis of the arm at select positions including: about 10 cm above the cubital fossa on the anterior side, about 5 cm and about 10 cm below the cubital fossa on the anterior side, about 10 cm above the olecranon, about 5 cm and about 10 cm below the olecranon, and on the dorsum of the hand. Tissue depth is determined using the ultrasound system. Thickness of the dermis is determined by measuring between the echo entry and the dermis/subcutis boundary, and the thickness of the subcutis is set between the bottom of the dermis and the fascial connective tissue sheet overlying the muscle. Statistical analysis of the differences in arm volume, dermal thickness and subcutis thickness can be performed using Statistical Package for the Social Sciences (SPSS Inc., Chicago, Ill., USA). A p-value <0.05 is considered to be statistically significant.

The Aloka SSD-1700 Diagnostic Ultrasound System (Aloka Co., Ltd. (Wallingford, Conn.) can also be used to characterize select portions of the upper and lower arm. For example, images are obtained at about 10 cm proximal and distal from the olecranon and shoulder and distal to the spine of the scapulae. From the images, dermal thickness, subcutaneous thickness and echogenicity are determined using software such as Adobe Photoshop 5.0 and by histogram analysis using the number of pixels at each brightness level in an image and the mean brightness value. Where needed, perimeter and skinfold measurement are obtained at about 15 cm proximal to the olecranon and about 10 cm distal to the olecranon using a Lange Skinfold Caliper (Beta Technology Inc., Santa Cruz, Calif., US). Data is tested for normality using a Wilcoxon test, and the differences between data points determine by a Student's t-test with significance at 5%.

Skin thickness of select portion(s) of the upper extremity can also be determined by ultrasound scanning using the DermaScan system (DermaScan C, Cortex Technology, Smedevaenget, Danmark) at 20 mHz or Acuson XP10 imager (Acuson, Mountain View, Calif., US) at 7 MHz. To assess the arm near the ulnar styloid process at the wrist, regions of about 13 cm to about 19 cm proximal to the ulnar styloid process are examined. The individual can be in a sitting position with the arm supported in abduction and then rotated and extended at the shoulder to position the ventral, lateral, dorsal and medial aspects uppermost. To determine epidermis, dermis and subcutis thickness, ultrasound scans are performed at 20 MHz using the DermaScan and 7 MHz using the Acuson XP10 imager at depth settings from about 2 cm to about 5 cm. Ultrasound gel is applied to select regions of the arm and then the ultrasound probe is placed transversely on the arm. To determine skin thickness (epidermis-dermis), a two-dimensional image of the skin about 2.3 cm deep is obtained and recorded using the DermaScan at 20 MHz. The typical field of view is about 13.4 mm wide to about 22.4 mm deep. To measure subcutis thickness, images of about 4 cm to about 5 cm deep are obtained using the Acuson XP 10 imager at a frequency of 7 MHz. The typical field of view is about 4.0 cm wide to about 4.0 or about 50 cm deep. Images are recorded using a video recorder, and thickness of the epidermis-dermis and subcutis are determined using image analysis software, e.g. Matlab, available from MathWorks Inc. (The MathWorks, Natick, Mass.). Skin thickness is measured between the bottom edge of the entry echo and the dermis/subcutis boundary, and subcutis thickness is measured between the bottom of the dermis to a line representing the fascial connective tissue sheath overlying the muscle, deep fascia or the lower complete line where double echo lines are obtained. Means values are determined using values from three images for each select region. The means±standard deviation (SD) are computed. Results are tested for Gaussian distribution using the Kolmogorov-Smirnov test, p>0.10 indicating normality. Variations between select regions in each arm (i.e., ventral, lateral, medial, and dorsal thicknesses in the contralateral arm) are tested using one-way analysis of variance (ANOVA) with Bonferroni multiple post comparison tests. Two-way ANOVA is applied to the results from the different sites and/or the different extremities (i.e., contralateral versus ipsilateral). Two-way ANOVA is also applied to results representing the degree of increase in thickness and patient variability. Other comparisons are made using Student's paired t-test. Correlations are tested using regression analysis. A p-value <0.05 is considered significant.

Skin thickness of select portion(s) of the lower extremity of an individual can be determined by ultrasound scanning using real-time 20-MHz high-resolution ultrasound imaging. The Dermcup 2020 (Atys Me'dica, Soucieu en Jarrest, France) is used to obtain images of about 5 cm deep with axial resolution of 80 μm and lateral resolution of 200 μm at an acquisition speed of 15 frames s⁻¹. The typical field of view is about 6 cm wide by 5 cm deep. To obtain the images of the skin, standard echographic gel is applied between the skin surface and the probe, and the probe is maintained perpendicular to the skin surface with minimal pressure applied to preserve the thickness and echogenicity of the skin. Images are obtained of three different sites for each lower limb: thigh (front of the thigh, halfway between the iliac spine and the knees), lower leg (lateral external side of the leg, halfway between the knee and the malleolus) and ankle (area just above the malleolus externally). Thickness is measured perpendicular to the skin surface from the skin surface to the deepest point of dermal echogenicity using an electronic calliper. At least three sonometric thicknesses are measured for each site, and the mean depth is used. Where the lower boundary of the dermis is unclear, the gain is increased until the boundary is easily identified. Images are recorded with a gain between 22 and 24 dB. Statistical analysis where needed included nonparametric tests (Wilcoxon test) using Epi Info software (http://www.cdc.gov/epiinfo/), and p<0.05 is considered significant.

### Other Embodiments of the Invention

While the invention has been described in conjunction with the detailed description, the foregoing description is intended to illustrate and not limit the scope of the invention, which is defined by the scope of the claims. Other aspects, advantages, and modifications are within the scope of the following claims.

The terms and expressions that have been employed are used as terms of description and not of limitation, and there is no intent in the use of such terms and expressions to exclude any equivalent of the features shown and described or portions thereof. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims. In addition, the invention has been described broadly and generically herein. Each of the narrower species and subgeneric groupings falling within the generic disclosure also form part of the invention.

As used herein and in the appended claims, the singular forms “a,” “an,” and “the” include plural reference unless the context clearly dictates otherwise. Under no circumstances may the patent application be interpreted to be limited to the specific examples or embodiments or methods specifically disclosed herein.

What is claimed is:

1. A method for detecting lymphedema in an individual comprising:
(a) using a light sensing device that comprises a video camera, depth sensor and software for generating a three-dimensional digital model of at least a portion of the body of the individual from data obtained by the camera and depth sensor to obtain a three-dimensional digital model of at least a portion of the body of the individual;
(b) determining the size of a first select region of the body from the three-dimensional digital model; and
(c) utilizing the size of the first select region and the size of a first reference region similarly determined according to steps (a) and (b) to obtain a size differential, wherein lymphedema is detected in the first select region if the size differential indicates that the size of the first select region is greater than the size of the first reference region by at least about 3%.

2. The method of claim 1, wherein the light sensing device comprises a RGB video camera and a charge-coupled device (CCD) image sensor, contact image sensor (CIS), or an active pixel sensor in complementary metal-oxide-semiconductor (CMOS).

3. The method of claim 1, wherein the light sensing device is a mobile device comprising software for generating, in real time, a three-dimensional model of at least a portion of the body of an individual.

4. The method of claim 1, wherein the sizes of the first select region and first reference region are indicated by the width, circumference or volume of the regions.

5. The method of claim 1, wherein the first select region and first reference region comprise symmetrical portions of the left and the right extremities.

6. The method of claim 5, wherein the size differential correspond to the difference between the size of the first select region and the size of the first reference region.

7. The method of claim 1, wherein the first select region and first reference region comprise substantially the same portion of the body occurring at a post-surgery and a pre-surgery time point, respectively.

8. The method of claim 7, further comprising determining the individual's weight at the post-surgery time point, and further utilizing the individual's weight at the post-surgery time point and a reference weight of the individual similarly determined at the pre-surgery time point to obtain a weight adjusted size differential.

9. The method of claim 7, further comprising determining the size of a second select region of the body from the three-dimensional digital model, wherein the first and second select regions comprise symmetrical portions of the left and the right extremities, and further utilizing the size of the second select region and a similarly determined size of a second reference region to obtain a size differential that corresponds to a relative volume change, wherein the second reference region corresponds substantially to the same portion of the body as the second select region and occurring at the pre-surgery time point.

10. The method of claim 1, wherein the first select region and first reference region comprise substantially the same region of the body occurring two time points post surgery, a later time point and an earlier time point, respectively.

11. A method for detecting lymphedema in an individual comprising:
(a) determining a size differential for a first select region and a first reference region according to claim 1, and
(b) determining the amount of extracellular fluids in the first select region of the body of the individual using bioimpedance spectroscopy, and comparing the amount of extracellular fluids in the first select region with the amount of extracellular fluids in the first reference region similarly determined using bioimpedance spectroscopy to obtain an impedance ratio change,
wherein lymphedema is detected in the first select region if the size differential indicates that the size of the first select region is greater than the size of the first reference region by at least about 3%, and the impedance ratio change is about 0.1 or more.

12. A method for detecting lymphedema in an individual comprising:
(a) determining a weight adjusted size differential for a first select region and a first reference region according to claim 8, and
(b) determining the amount of extracellular fluids in the first select region of the body of the individual using bioimpedance spectroscopy, and comparing the amount of extracellular fluids in the first select region with the amount of extracellular fluids in the first reference region similarly determined using bioimpedance spectroscopy to obtain an impedance ratio change,
wherein lymphedema is detected in the first select region if the weight adjusted size differential indicates that the size of the first select region is greater than the size of the first reference region by at least about 3%, and the impedance ratio change is about 0.1 or more.

13. A method for detecting lymphedema in an individual comprising:
(a) determining a size differential corresponding to relative volume change according to claim 9, and
(b) determining the amount of extracellular fluids in the first select region of the body of the individual using bioimpedance spectroscopy, and comparing the amount of extracellular fluids in the first select region with the amount of extracellular fluids in the second select region similarly determined using bioimpedance spectroscopy to obtain an impedance ratio change,
wherein lymphedema is detected in the first select region if the size differential indicates that the size of the first select region is greater than the size of the first reference region by at least about 3%, and the impedance ratio change is about 0.1 or more.

14. A method for detecting lymphedema in an individual comprising:
(a) determining a size differential for a first select region and a first reference region according to claim 1, and
(b) determining skin thickness in the first select region using ultrasound, and comparing the thickness of the skin in the first select region with thickness of the skin in the first reference region similarly determined using ultrasound to obtain an impedance ratio change,
wherein lymphedema is detected in the first select region if the size differential indicates that the size of the first select region is greater than the size of the first reference region by at least about 3%, and thickness of the skin is increased by at least about 5%.

15. The method of claim 14, wherein skin thickness is indicated by epidermis-dermis depth.

16. The method of claim 14, wherein skin thickness is indicated by subcutis depth.

17. A method for detecting lymphedema in an individual comprising:
(a) Determining the amount of extracellular fluids and skin thickness at a select region of the body of the individual, wherein amount of extracellular fluids is determined using bioimpedance spectroscopy, and thickness of the skin is determined using ultrasound; and
(b) Comparing the amount of extracellular fluids and skin thickness at the select region to that of a reference region similarly determined according to step (a), wherein early-stage lymphedema is detected in the select region if the increase in amount of extracellular fluids in the select region relative to the reference region corresponds to an impedance ratio change of about 0.1 or more, and the skin thickness at the select region relative to the reference region is increased by at least about 5%.

18. A method for managing lymphedema in an individual comprising monitoring for lymphedema in an individual at risk for lymphedema using the method of claim 1 and providing medical guidance or treatment to manage lymphedema in the individual.

19. A method for managing lymphedema in an individual comprising monitoring for lymphedema in an individual at risk for lymphedema using the method of claim 11 and providing medical guidance or treatment to manage lymphedema in the individual.

20. A method for managing lymphedema in an individual comprising monitoring for lymphedema in an individual at risk for lymphedema using the method of claim 14 and providing medical guidance or treatment to manage lymphedema in the individual.

21. A method for managing lymphedema in an individual comprising monitoring for lymphedema in an individual at risk for lymphedema using the method of claim 17 and providing medical guidance or treatment to manage lymphedema in the individual.
